(19) United States
(12) Patent Application Publication
(10) Pub. No.: US 2026/0102426 A1
(43) Pub. Date: Apr. 16, 2026

(54) METHOD OF ACTIVATING MITOCHONDRION

(71) Applicants: Chung-Tai Chang, Tainan City (TW); Chia-Hao Chang, Tainan City (TW); Ting-Yi Chang, Tainan City (TW)

(72) Inventors: Chung-Tai Chang, Tainan City (TW); Chia-Hao Chang, Tainan City (TW); Ting-Yi Chang, Tainan City (TW)

(21) Appl. No.: 18/912,460

(22) Filed: Oct. 10, 2024

(51) Int. Cl. A61K 33/06; A61K 33/00

(52) U.S. Cl. CPC A61K 33/06; A61K 33/00

(57) ABSTRACT

The present invention relates to a method of activating mitochondrion, primarily to provide a negative charge and reactive oxygen to the mitochondrion from outside via a negative charge and reactive oxygen supply unit to be as a material for mitochondrion redox. A calcium ion channel stimulating unit stimulates an in vivo calcium ion to transfer the protein into an matrix space of the mitochondrion through the calcium on the inner membrane of the mitochondrion, so that the inflow of calcium ion can promote the depolarization of the mitochondrion and increase the oxidative phosphorylation activity of the mitochondrion. Therefore, the mitochondrion activation and ATP generation are enhanced.

### BACKGROUND OF THE INVENTION

The present invention relates to a method of activating mitochondrion, and more particularly to supply negative charges (−e) and reactive oxygen (O) to the mitochondrion from ex vivo by negative charge and reactive oxygen supply unit, and stimulate in vivo calcium ions to pass through the calcium on the endomembrane of mitochondrion to transfer protein into the matrix space of the mitochondrion via stimulating unit of a calcium ion channel, so as to enhance activation of mitochondrion and generation of adenosine triphosphate (ATP).

### DESCRIPTION OF THE RELATED ART

Accordingly, the mitochondrion is a unit generating energy in the cell, namely, an energy factory of the cell. Each mitochondrion is composed of a layer of an outer membrane and a layer of an inner membrane and the space surrounded by the inner membrane is called matrix space. The outer membrane of a mitochondrion is embedded with enzymes related to biological oxidation and supply materials required for reaction within the mitochondrion. In the mitochondrion, the matrix space contains some enzymes capable of metabolizing and conversing carbohydrates, proteins and fats to H2O and CO2 through the citric acid cycle. During the process, the electrons are transmitted via electron transport chain and synthesized to be adenosine triphosphate (ATP) through oxidative phosphorylation and ATP is the main source of energy of energy consuming reactions in plants and animals. ATP is a kind of nucleotide in biology used as the energy currency for intracellular energy transfer to store and transmit chemical energy. ATP has an important function on nucleic acid synthesis. Further, ATP is the disodium guanylate in RNA and acts as a substitute during DNA transcription.

However, the continuous reduction-oxidation is required for the mitochondrion to produce energy. The activating power of reduction-oxidation relies on negative charge (−e) and reactive oxygen (O), wherein the negative charge (−e) and reactive oxygen (O) are the materials for mitochondrion to function. If the above materials can be supplied to the mitochondrion, the mitochondrion can be more active. In addition, the inflow of calcium ions has important functions on the mitochondrion activation and ATP generation as followings:
1. Controlling of calcium ions within the mitochondrion: calcium ions can enter the mitochondrion and affect its function, when the calcium level in the cytoplasm is rose, the calcium ions transports protein into the matrix space of the mitochondrion via the calcium on the inner membrane of the mitochondrion. The inflow of calcium ions can promote depolarization of the mitochondrion to enhance the activation of oxidative phosphorylation of the mitochondrion.
2. Promoting the oxidative phosphorylation: the increase of calcium ions enables to activate a various kinds of enzyme systems within the mitochondrion, such as the key enzyme in tricarboxylic acid cycle (TCA). The activation of these enzymes enables mitochondrion to accelerate the oxidation process of the substrate (also called zymolyte or functioning substance) and enhances the efficiency of the electron transport chain to finally contribute to the generation of ATP.
3. Increasing the ATP synthesis: when the calcium level in the mitochondrion is rose, the tricarboxylic acid cycle (TCA cycle) and oxidative phosphorylation are accelerated. The above-mentioned oxidative phosphorylation is a kind of metabolic processing of the cell and the process occurs in the inner membrane of the mitochondrion of eukaryote or the cell membrane of prokaryote. The enzymes and the energy released from the oxidation of various nutrients are used to synthesize ATP which directly increase the generation of ATP. The generation of ATP is driven by the operation of the electron transport chain and the processes promoted by calcium ions ensure the continuity and efficiency of ATP generation.
4. Maintaining the energy balance of the cell: the increasing ATP generation is beneficial to maintain the energy balance of the cell, especially when facing various pressures. This also benefits the repair of the cell and recovery to normal function, thereby avoiding death of the cell due to insufficient energy.

Therefore, if negative charges (−e) and reactive oxygen (O) required for mitochondrion redox are continuously supplied from the outside and promoting in vivo calcium ions to transport protein into the matrix space of the mitochondrion through the calcium on the inner membrane of the mitochondrion, thereby enhancing the mitochondrion activation and ATP generation.

### SUMMARY OF THE INVENTION

It is a primary objective of the present invention to provide a method of activating mitochondrion capable of enhancing the mitochondrion activation and ATP generation.

The primary feature of the present invention is related to step 1 of that suppling negative charges and reactive oxygen from outside to the mitochondrion through the negative charges and reactive oxygen supply units to be as the material of mitochondrion redox, and step 2 of that the prompting unit of calcium ion channel enabling to promote in vivo calcium ion to transport protein into the matrix space of the mitochondrion channel through the calcium on the inner membrane of the mitochondrion so that the inflow of calcium ions can promote depolarization of the mitochondrion and increase the activation of oxidative phosphorylation of the mitochondrion, thereby enhancing mitochondrion activation and ATP generation.

For better understanding of the technical means to accomplish the above object and the effect of the present invention, detailed descriptions are provided below with respect to the following embodiment.

### BRIEF DESCRIPTION OF THE DRAWINGS

FIG. 1 is a block diagram of a preferred embodiment of the present invention.

Appendix is related to a test report of Taiwan Mitochondrion Applied Technology Co., Ltd.

### DETAILED DESCRIPTION OF EMBODIMENTS OF THE INVENTION

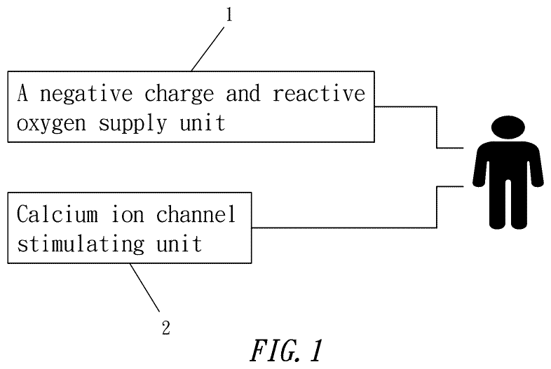

With reference to FIG. 1, the method of activating mitochondrion according to a preferred embodiment of the present invention are as followings:
Step 1 is to provide negative charge (−e) and reactive oxygen (O) to the mitochondrion. Through the negative charge and reactive oxygen supply unit 1, the negative charges (−e) and reactive oxygen (O) are provided to the mitochondria from outside to be the material of mitochondrion redox. The negative charge and reactive oxygen supply unit 1 can be implemented and achieved by an electrified low temperature plasma generator. After being electrified, the low temperature plasma generator will generate a large quantity of negative charge (−e) and reactive oxygen (O) and the large quantity of negative charge (−e) and reactive oxygen (O) will enter into the interior of human body to be the material of mitochondrion redox.
Step 2 is to provide calcium ions to the mitochondrion. The calcium ion channel stimulating unit 2 stimulates the in vivo calcium ions to transfer the protein into the matrix space of the mitochondrion through the calcium on the inner membrane of the mitochondrion, so that the inflow of calcium ions can promote depolarization of the mitochondrion. The calcium ion channel stimulating unit 2 can be a far infrared generator having power number above 500 W and the body being radiated by the powered far infrared generator can stimulate the in vivo calcium ions to transfer protein into the matrix space of the mitochondrion through the calcium on the inner membrane of the mitochondrion The negative charge and reactive oxygen supply unit 1 of the above-mentioned Step 1 is to provide the sufficient material of negative charge (−e) and reactive oxygen (O) to the mitochondrion and then the calcium ion channel stimulating unit 2 of Step 2 is to stimulate the in vivo calcium ions to transfer the protein into the matrix space of the mitochondrion through the calcium on the inner membrane of mitochondrion, so that the inflow of calcium ions can promote depolarization of the mitochondrion and enhance mitochondrion activation and ATP generation.

Moreover, Taiwan Mitochondrion Applied Technology Co., Ltd. tested the mitochondrial activation of peripheral blood mononuclear cell (PBMC) of testee A, B and C individually before using the present invention, one hour after second using, and one hour after the fourth using. The test results are as shown in the first, second, and third test reports in the appendix. According to the test results, after application of the present invention, the mitochondrion is obviously activated. As proved by the test results, the present invention is truly effective and has value for industrial application.

In summary, the present invention not only has novelty, but also has value for industrial utilization. Therefore, an application for invention patent is submitted. Your examination and approval will be highly appreciated.

What is claimed is:

1. A method of activating mitochondrion, comprising
step 1, suppling a negative charge and the reactive oxygen to a mitochondrion, the negative charge and the reactive oxygen being supplied from outside to the mitochondrion via a negative charge and reactive oxygen supply unit to be as a material for mitochondrion redox; and,
step 2, suppling a calcium ion to the mitochondrion, a calcium ion channel stimulating unit stimulating an in vivo calcium ion to transfer a protein into a matrix space of the mitochondrion through a calcium on an inner membrane of the mitochondrion, thereby enabling an inflow of the calcium ion to promote a depolarization of the mitochondrion and increasing an oxidative phosphorylation activity of the mitochondrion.

2. The method of activating mitochondrion of claim 1, wherein the negative charge and reactive oxygen supply unit is a low temperature plasma generator.

3. The method of activating mitochondrion of claim 1, wherein the calcium ion channel stimulating unit is a far infrared generator having a power number above 500 W.
